SEQUENCE LISTING (89 sequences)

SEQ ID NO: 1 - protein, 17 aa, Artificial Sequence
  The sequence is synthesized.
PPDDMYYYCDYKDDDDK

SEQ ID NO: 2 - RNA, 42 nt, Artificial Sequence
  The sequence is synthesized.
auguacuacuaccaugacuacaaggacgacgacgacaaguaa

SEQ ID NO: 3 - DNA, 21 bp, Artificial Sequence
  mRNA 3′UTR
cccgcctcccgccccccgtcc

SEQ ID NO: 4 - protein, 15 aa, Artificial Sequence
  The sequence is synthesized.
RWRFYSGPYFILAAC

SEQ ID NO: 5 - protein, 15 aa, Artificial Sequence
  The sequence is synthesized.
RWRLYSGPYFILAAC

SEQ ID NO: 6 - protein, 15 aa, Artificial Sequence
  The sequence is synthesized.
RWRFYSGPYFILASC

SEQ ID NO: 7 - protein, 15 aa, Artificial Sequence
  The sequence is synthesized.
RWRFYSGPYFILALC

SEQ ID NO: 8 - protein, 15 aa, Artificial Sequence
  The sequence is synthesized.
GWRFYSGPYFILAVC

SEQ ID NO: 9 - protein, 15 aa, Artificial Sequence
  The sequence is synthesized.
VWRLYSGPYFILAVC

SEQ ID NO: 10 - protein, 16 aa, Artificial Sequence
  The sequence is synthesized.
VWRLYSGPYFTLALSC

SEQ ID NO: 11 - protein, 16 aa, Artificial Sequence
  The sequence is synthesized.
DWRFYSGPYFTLAWAC

SEQ ID NO: 12 - protein, 14 aa, Artificial Sequence
  The sequence is synthesized.
GVKRIYVVYVLSVC

SEQ ID NO: 13 - protein, 16 aa, Artificial Sequence
  The sequence is synthesized.
DGPLFLVLVVFRSSAC

SEQ ID NO: 14 - protein, 16 aa, Artificial Sequence
  The sequence is synthesized.
RGHCWLYVYFPVRSLC

SEQ ID NO: 15 - protein, 16 aa, Artificial Sequence
  The sequence is synthesized.
GGHCWLYVYFPVRSAC

SEQ ID NO: 16 - protein, 16 aa, Artificial Sequence
  The sequence is synthesized.
RGHCWLYVYFPVRSVC

SEQ ID NO: 17 - protein, 16 aa, Artificial Sequence
  The sequence is synthesized.
LGHCWLYVYFPVRSVC

SEQ ID NO: 18 - protein, 16 aa, Artificial Sequence
  The sequence is synthesized.
VGHCWLYVYFPVRSLC

SEQ ID NO: 19 - protein, 16 aa, Artificial Sequence
  The sequence is synthesized.
VGHCWLYVYFPVCSLC

SEQ ID NO: 20 - protein, 16 aa, Artificial Sequence
  The sequence is synthesized.
RGHCWLYVYFPVCSLC

SEQ ID NO: 21 - protein, 14 aa, Artificial Sequence
  The sequence is synthesized.
RAVIILYWRIPSLC

SEQ ID NO: 22 - protein, 14 aa, Artificial Sequence
  The sequence is synthesized.
NAYSFWLYLRFPSC

SEQ ID NO: 23 - protein, 14 aa, Artificial Sequence
  The sequence is synthesized.
GVIWIYFIRKRSVC

SEQ ID NO: 24 - protein, 15 aa, Artificial Sequence
  The sequence is synthesized.
VGCVFVFVFYPQSSC

SEQ ID NO: 25 - protein, 15 aa, Artificial Sequence
  The sequence is synthesized.
HVRIYVYFLTRTPAC

SEQ ID NO: 26 - protein, 15 aa, Artificial Sequence
  The sequence is synthesized.
VGSRILCLIFLLRVV

SEQ ID NO: 27 - protein, 16 aa, Artificial Sequence
  The sequence is synthesized.
RGHCWLYVYFPVRSLS

SEQ ID NO: 28 - protein, 15 aa, Artificial Sequence
  The sequence is synthesized.
RWRFYSGMRNREMDC

SEQ ID NO: 29 - protein, 15 aa, Artificial Sequence
  The sequence is synthesized.
RWRFYSGHKQEMDSC

SEQ ID NO: 30 - protein, 15 aa, Artificial Sequence
  The sequence is synthesized.
RWRFYSGGTKEMEYC

SEQ ID NO: 31 - protein, 15 aa, Artificial Sequence
  The sequence is synthesized.
RWRFYSGMRNREMAC

SEQ ID NO: 32 - protein, 15 aa, Artificial Sequence
  The sequence is synthesized.  Xaa can be any naturally occurring amino acid.
  misc_feature (11)..(11): Xaa can be any naturally occurring amino acid
RWRFYSGMRKXEMDC

SEQ ID NO: 33 - protein, 15 aa, Artificial Sequence
  The sequence is synthesized.
RWRFYSGRQKEMDVC

SEQ ID NO: 34 - protein, 15 aa, Artificial Sequence
  The sequence is synthesized.
RWRFYSGEPEREMAC

SEQ ID NO: 35 - protein, 15 aa, Artificial Sequence
  The sequence is synthesized.
RWRFYSGIRNREIDC

SEQ ID NO: 36 - DNA, 45 bp, Artificial Sequence
  The sequence is synthesized.
cgttggcgtttttatagtggtccgtattttattctggcggcgtgc

SEQ ID NO: 37 - DNA, 45 bp, Artificial Sequence
  The sequence is synthesized.
cgttggcgtctttatagtggtccgtattttattctggcggcgtgc

SEQ ID NO: 38 - DNA, 45 bp, Artificial Sequence
  The sequence is synthesized.
cgttggcgtttttatagtggtccgtattttattctggcgtcgtgc

SEQ ID NO: 39 - DNA, 45 bp, Artificial Sequence
  The sequence is synthesized.
cgttggcgtttttatagtggtccgtattttattctggcgttgtgc

SEQ ID NO: 40 - DNA, 45 bp, Artificial Sequence
  The sequence is synthesized.
ggttggcgtttttatagtggtccgtattttattctggcggtgtgc

SEQ ID NO: 41 - DNA, 45 bp, Artificial Sequence
  The sequence is synthesized.
gtttggcgtctttatagtggtccgtattttattctggcggtgtgc

SEQ ID NO: 42 - DNA, 48 bp, Artificial Sequence
  The sequence is synthesized.
gtttggcgtctttatagtggtccgtattttactctggcgttgtcgtgc

SEQ ID NO: 43 - DNA, 48 bp, Artificial Sequence
  The sequence is synthesized.
gattggcgtttttatagtggtccgtattttactctggcgtgggcgtgc

SEQ ID NO: 44 - DNA, 42 bp, Artificial Sequence
  The sequence is synthesized.
ggtgtgaagcgtatttatgtggtttatgttctttcggtgtgc

SEQ ID NO: 45 - DNA, 48 bp, Artificial Sequence
  The sequence is synthesized.
gatggtccgttgtttcttgttttggttgtgtttcgttcttcggcgtgc

SEQ ID NO: 46 - DNA, 48 bp, Artificial Sequence
  The sequence is synthesized.
cgtggtcattgttggttgtatgtgtattttcctgttcgttcgttgtgc

SEQ ID NO: 47 - DNA, 48 bp, Artificial Sequence
  The sequence is synthesized.
ggtggtcattgttggttgtatgtgtattttcctgtccgttcggcgtgc

SEQ ID NO: 48 - DNA, 48 bp, Artificial Sequence
  The sequence is synthesized.
cgtggccattgttggttgtatgtgtattttcctgttcgttcggtgtgc

SEQ ID NO: 49 - DNA, 48 bp, Artificial Sequence
  The sequence is synthesized.
cttggtcattgttggttgtatgtgtattttcctgttcgttcggtgtgc

SEQ ID NO: 50 - DNA, 48 bp, Artificial Sequence
  The sequence is synthesized.
gttggtcattgttggttgtatgtgtattttcctgttcgttcgttgtgc

SEQ ID NO: 51 - DNA, 48 bp, Artificial Sequence
  The sequence is synthesized.
gttggtcattgttggttgtatgtgtattttcctgtttgttcgttgtgc

SEQ ID NO: 52 - DNA, 48 bp, Artificial Sequence
  The sequence is synthesized.
cgtggtcattgttggttgtatgtgtattttcctgtttgttcgttgtgc

SEQ ID NO: 53 - DNA, 42 bp, Artificial Sequence
  The sequence is synthesized.
cgtgctgtgattattctgtattggaggattccttcgttgtgc

SEQ ID NO: 54 - DNA, 42 bp, Artificial Sequence
  The sequence is synthesized.
aatgcgtatagtttttggctgtatcttcgttttccgtcgtgc

SEQ ID NO: 55 - DNA, 42 bp, Artificial Sequence
  The sequence is synthesized.
ggtgttatttggatttattttattcgtaagcgttcggtgtgc

SEQ ID NO: 56 - DNA, 45 bp, Artificial Sequence
  The sequence is synthesized.
gttggttgtgtttttgtttttgtgttttatcctcagtcgtcgtgc

SEQ ID NO: 57 - DNA, 45 bp, Artificial Sequence
  The sequence is synthesized.
catgtgcgtatttatgtgtattttttgactcgtactccggcgtgc

SEQ ID NO: 58 - DNA, 46 bp, Artificial Sequence
  The sequence is synthesized.
gttggcagcagaatcttgtgtctgatcttccttttacgcgttgtgc

SEQ ID NO: 59 - DNA, 45 bp, Artificial Sequence
  The sequence is synthesized.
cgttggcgtttctactccggcatgcggaatagggagatggattgc

SEQ ID NO: 60 - DNA, 45 bp, Artificial Sequence
  The sequence is synthesized.
cgttggcgtttctactccggccataagcaggagatggattcgtgc

SEQ ID NO: 61 - DNA, 45 bp, Artificial Sequence
  The sequence is synthesized.
cgttggcgtttctactccggcggtacgaaggagatggagtattgc

SEQ ID NO: 62 - DNA, 45 bp, Artificial Sequence
  The sequence is synthesized.
cgttggcgtttctactccggcatgcggaatagggagatggcatgc

SEQ ID NO: 63 - DNA, 45 bp, Artificial Sequence
  The sequence is synthesized.
cgttggcgtttctactccggcatgcgtaagtaggagatggattgc

SEQ ID NO: 64 - DNA, 45 bp, Artificial Sequence
  The sequence is synthesized.
cgttggcgtttctactccggccggcagaaggagatggatgtgtgc

SEQ ID NO: 65 - DNA, 45 bp, Artificial Sequence
  The sequence is synthesized.
cgttggcgtttctactccggcgagcctgagagggagatggcttgc

SEQ ID NO: 66 - protein, 17 aa, Artificial Sequence
  The sequence is synthesized.
LLLMWCAVLVVLRWRRR

SEQ ID NO: 67 - protein, 17 aa, Artificial Sequence
  The sequence is synthesized.
LLLMWCAVLVVVRWRRR

SEQ ID NO: 68 - protein, 16 aa, Artificial Sequence
  The sequence is synthesized.
AFLLLWSHGRPRIIFC

SEQ ID NO: 69 - protein, 9 aa, Artificial Sequence
  The sequence is synthesized.
HSPFTFQHC

SEQ ID NO: 70 - protein, 9 aa, Artificial Sequence
  The sequence is synthesized.
HSPFTFQQC

SEQ ID NO: 71 - protein, 8 aa, Artificial Sequence
  The sequence is synthesized.
HSPFTFSS

SEQ ID NO: 72 - protein, 11 aa, Artificial Sequence
  The sequence is synthesized.
PSSCHTWHRLC

SEQ ID NO: 73 - protein, 11 aa, Artificial Sequence
  The sequence is synthesized.
PSSRHTWHRWV

SEQ ID NO: 74 - protein, 10 aa, Artificial Sequence
  The sequence is synthesized.
PSSCHTWHRL

SEQ ID NO: 75 - protein, 10 aa, Artificial Sequence
  The sequence is synthesized.
HECSHRPTEC

SEQ ID NO: 76 - protein, 11 aa, Artificial Sequence
  The sequence is synthesized.
FHLRARSLHTC

SEQ ID NO: 77 - protein, 12 aa, Artificial Sequence
  The sequence is synthesized.
LLLMWCAVLVVV

SEQ ID NO: 78 - DNA, 51 bp, Artificial Sequence
  The sequence is synthesized.
ttgcttctgatgtggtgtgcagtcttagtcgtattgcggtggaggaggagg

SEQ ID NO: 79 - DNA, 51 bp, Artificial Sequence
  The sequence is synthesized.
ttgcttctgatgtggtgtgcagtcttagtcgtagtgcggtggaggaggagg

SEQ ID NO: 80 - DNA, 48 bp, Artificial Sequence
  The sequence is synthesized.
gcgtttttgctgttgtggtctcatgggaggccgaggattattttttgc

SEQ ID NO: 81 - DNA, 27 bp, Artificial Sequence
  The sequence is synthesized.
cattctccgtttacgttttagcattgc

SEQ ID NO: 82 - DNA, 27 bp, Artificial Sequence
  The sequence is synthesized.
cattctccgtttacgttttagcagtgc

SEQ ID NO: 83 - DNA, 26 bp, Artificial Sequence
  The sequence is synthesized.
cattctccgtttacgtttagcagtgc

SEQ ID NO: 84 - DNA, 32 bp, Artificial Sequence
  The sequence is synthesized.
ccttcgtcgtgtcatacgtggcatcgtttgtg

SEQ ID NO: 85 - DNA, 34 bp, Artificial Sequence
  The sequence is synthesized.
ccttcgtcgcgtcatacgtggcatcgttgggtgc

SEQ ID NO: 86 - DNA, 31 bp, Artificial Sequence
  The sequence is synthesized.
ccttcgtcgtgtcatacgtggcatcgtttgc

SEQ ID NO: 87 - DNA, 30 bp, Artificial Sequence
  The sequence is synthesized.
catgagtgttcgcatcgtccgactttttgc

SEQ ID NO: 88 - DNA, 33 bp, Artificial Sequence
  The sequence is synthesized.
tttcatcttcgtgcgcggagtcttcatacgtgc

SEQ ID NO: 89 - DNA, 36 bp, Artificial Sequence
  The sequence is synthesized.
ttgcttctgatgtggtgtgcagtcttagtcgtagtc

(19) United States
(12) Patent Application Publication
(10) Pub. No.: US 2025/0026791 A1
(43) Pub. Date: Jan. 23, 2025

(54) CYCLIC PEPTIDE CYCLIZED IN AROMATIC NUCLEOPHILIC SUBSTITUTION REACTION, AND PEPTIDE LIBRARY, PRODUCTION METHOD, AND SCREENING METHOD FOR SAME

(71) Applicant: UNIVERSITY OF YAMANASHI, Kofu-shi, Yamanashi (JP)

(72) Inventor: Takashi KAWAKAMI, Kofu-shi (JP)

(73) Assignee: UNIVERSITY OF YAMANASHI, Kofu-shi, Yamanashi (JP)

(21) Appl. No.: 18/571,941

(22) PCT Filed: Jun. 16, 2022
(86) PCT No.: PCT/JP2022/024227

§ 371 Date: Aug. 26, 2024

(30) Foreign Application Priority Data
Jun. 24, 2021 (JP) 2021-105191

(51) Int. Cl. C07K 7/54; A61K 38/00; C12N 15/11

(52) U.S. Cl. CPC C07K 7/54; C12N 15/11; A61K 38/00

(57) ABSTRACT

A conventional art had the problems that a mixture of a plurality of cyclic peptides can be formed and the cyclization of a peptide does not spontaneously proceed. The present invention provides a peptide compound having a cyclic portion, wherein the cyclic portion has a benzoic acid derivative linker cyclized by an aromatic nucleophilic substitution reaction and a peptide backbone, the peptide backbone has a residue with a thiol group, and the benzoic acid derivative linker is bonded to the peptide backbone via an N-terminal amino acid residue of the peptide backbone and the residue with a thiol group.

### TECHNICAL FIELD

This invention relates to cyclic peptides, in particular to cyclic peptide cyclized in aromatic nucleophilic substitution reaction, and peptide library, production method, and screening method for same.

### BACKGROUND

Existing pharmaceuticals are mainly classified into two categories: low molecular weight drugs and antibody drugs (high molecular weight drugs). In comparison with the antibody drugs (high molecular weight drugs), the low molecular drugs have lower pharmacological activity, are more likely to cause side effects, and have difficulty in inhibiting protein-protein interactions.

On the other hand, in comparison with the low molecular weight drugs, the antibody drugs (high molecular weight drugs) have problems such as high cost, inability to be administered orally, and the need for refrigerated and frozen storage.

Medium molecular drugs exist as a solution to the problems in both drugs. Peptide drugs, one of the middle molecular drugs, have begun to attract attention in recent years from many pharmaceutical companies, including Chugai, Shionogi, BMS, Novartis, and Merck.

There are two types of the peptide drugs: linear peptides and cyclized peptides. Since the linear peptides are unstable in vivo, the cyclized peptides are emphasized in the development of the peptide drugs.

Patent Documents 1 to 3 disclose conventional techniques for cyclizing peptides by nucleophilic alkyl substitution reactions.

### PRIOR ARTS

### Patent Documents

Patent Document 1: JP5605602B
Patent Document 2: JP6004399B
Patent Document 3: JP6294080B

### SUMMARY OF THE INVENTION

### Problem to be Solved by the Invention

In the techniques disclosed by the Patent Documents 1 and 2, peptides are cyclized not only with thiol groups present in cysteine residues and the like, but also with imidazole groups present in histidine residues and the like. There is a problem that this may form a mixture of multiple cyclized peptides.

In the technique disclosed by Patent Document 3, peptide cyclization does not proceed spontaneously. Therefore, additional operations for the peptide cyclization are required.

### Means for Solving the Problem

The inventor has developed a method for cyclizing peptides in a thiol group-selective manner, and a method in which peptide cyclization proceeds spontaneously, without the need for additional operations for the peptide cyclization.

The purpose of the invention is to provide a peptide compound having a cyclic moiety,
in which the cyclic moiety has:
   a benzoic acid derivative linker to be cyclized by an aromatic nucleophilic substitution reaction; and
   a peptide backbone,
the peptide backbone has a thiol group-containing residue, and
the benzoic acid derivative linker is bound to the peptide backbone via an N-terminal amino acid residue of the peptide backbone and the thiol group-containing residue.

The peptide compound having the cyclic moiety is more stable in vivo than linear peptides, thus providing stable peptide drugs.

In addition, the benzoic acid derivative linker may be a compound represented by the following chemical formula (I):

### [Chemistry 8]

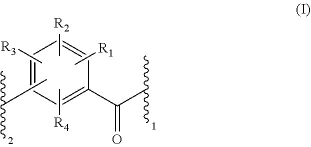

(in the chemical formula (I),
R₁, R₂, R₃ and R₄ are independently —F, —H, —Cl, —Br, —SO₂NH₂, —CF₃, —SO₂CH₃ or —NO₂, respectively,
a wave line 1 represents a covalent bond with the N-terminal amino acid residue of the peptide backbone, and
a wave line 2 represents a covalent bond with the thiol group-containing residue in the peptide backbone).

Another purpose of the invention is to provide a peptide compound having a cyclic moiety,
in which the cyclic moiety has:
   a benzoic acid derivative; and
   a peptide backbone,
the peptide backbone has a thiol group-containing residue,
the benzoic acid derivative linker is bound to the peptide backbone via an N-terminal amino acid residue of the peptide backbone and the thiol group-containing residue, and
the benzoic acid derivative linker is a compound represented by the following chemical formula (I):

### [Chemistry 9]

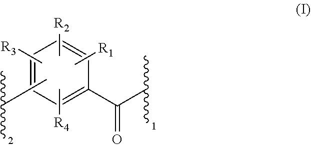

(in the chemical formula (I),
R₁, R₂, R₃ and R₄ are independently —F, —H, —Cl, —Br, —SO₂NH₂, —CF₃, —SO₂CH₃ or —NO₂, respectively,
a wave line 1 represents a covalent bond with the N-terminal amino acid residue of the peptide backbone, and
a wave line 2 represents a covalent bond with the thiol group-containing residue in the peptide backbone).

In addition, the benzoic acid derivative linker may be a compound represented by the following chemical formula (II) or (III):

### [Chemistry 10]

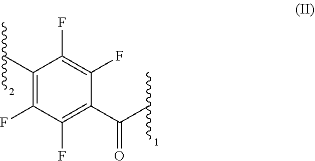 [SMILES: *C(=O)c1c(F)c(F)c(*)c(F)c1F]

### [Chemistry 11]

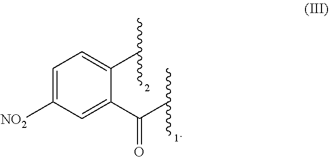 [SMILES: *C(=O)c1cc([N+](=O)[O-])ccc1*]

The peptide backbone may have an amino acid sequence described in any one of SEQ IDs: 4 to 35 and 66 to 77.

The thiol group-containing residue may be a cysteine residue.

Another purpose of the invention is to provide a pharmaceutical composition for treating or preventing a disease caused by a predetermined compound by binding the peptide compound to the predetermined compound.

The pharmaceutical composition including the peptide compound having the cyclic moiety is more stable in vivo than pharmaceutical compositions having linear peptides, thus providing stable peptide drugs.

The predetermined compound may be a bioactive protein. the bioactive protein may be PCSK9 or IL-5. the disease may be hypercholesterolemia or allergic disease.

Another purpose of the invention is to provide an initiator tRNA having a fluorobenzoic acid derivative linker precursor represented by the following chemical formula (IV):

### [Chemistry 12]

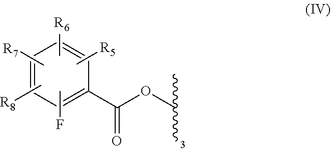

(In the chemical formula (IV),

R₅, R₆, R₇ and R₅ are independently —F, —H, —Cl, —Br, —SO₂NH₂, —CF₃, —SO₂CH₃ or —NO₂, respectively, and
a wave line 3 represents a covalent bond with tRNA).

In production of the peptide compound having the cyclic moiety using the initiator tRNA, the peptide is cyclized without cyclization treatment.

In addition, the fluorobenzoic acid derivative linker precursor may be a compound represented by the following chemical formula (V) or (VI):

### [Chemistry 13]

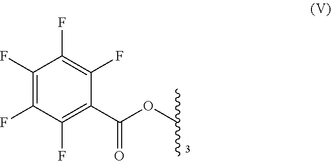 [SMILES: *OC(=O)c1c(F)c(F)c(F)c(F)c1F]

### [Chemistry 14]

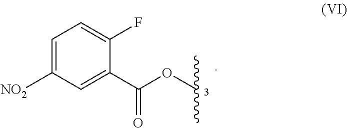 [SMILES: *OC(=O)c1cc([N+](=O)[O-])ccc1F]

Another purpose of the invention is to provide a method for producing the peptide compound, including:
a providing step for providing at least one type of mRNA; and
a translation step for translating the mRNA in a presence of the initiator tRNA,
in which the mRNA has an upstream nucleotide sequence having a nucleotide sequence corresponding to a start codon and a downstream nucleotide sequence having a nucleotide sequence encoding the peptide backbone.

The peptide compound having the cyclic moiety can be produced by this producing method. In this producing method, the peptide is cyclized without cyclization treatment.

Another purpose of the invention is to provide a method for screening a target protein, including:
a providing step for providing at least one type of mRNA;
a translation step for translating the mRNA in a presence of the initiator tRNA and a puromycin DNA linker to obtain a conjugate of a peptide compound having a cyclic moiety with a nucleic acid;
a contact step for contacting the conjugate with the target protein; and
an analysis step for analyzing a binding of the target protein and the conjugate;
in which the mRNA has an upstream nucleotide sequence having a nucleotide sequence corresponding to a start codon and a downstream nucleotide sequence having a nucleotide sequence encoding the peptide backbone.

This screening method can be used to screen a peptide compound having a cyclic moiety that bind to a target protein.

### BRIEF DESCRIPTION OF THE DRAWING

FIG. 1 shows a scheme for producing a cyclic peptide with FNO₂ Ph.

FIG. 2 shows a scheme for producing a cyclic peptide with F₅Ph.

FIG. 3 shows a MALDI-TOF (Matrix Assisted Laser Desorption/Ionization-Time-Flight) mass spectrometer chart for an analysis of the cyclic peptide with FNO₂Ph.

FIG. 4 shows a MALDI-TOF mass spectrometer chart for an analysis of the cyclic peptide with F₅ Ph

FIG. 5 shows a scheme for screening human PCSK9.

FIG. 6 shows a collection rate of cDNA for the cyclic peptide with FNO₂Ph.

FIG. 7 shows a collection rate of cDNA for the cyclic peptide with F₅Ph.

FIG. 8 shows a sequence of the collected cyclic peptide with FNO₂Ph.

FIG. 9 shows a sequence of the collected cyclic peptide with F₅Ph.

FIG. 10 shows a graph of a collection rate of binding cDNA/mRNA presenting the cyclic peptide with FNO₂Ph including an amino acid sequence of SEQ ID: 4 (Peptide 1) to PCSK9.

FIG. 11 shows a graph of a collection rate of binding cDNA/mRNA presenting cyclic peptide with F₅Ph having an amino acid sequence of SEQ ID: 14 (Peptide 2) to PCSK9.

FIG. 12 shows a graph of collection rates of binding Peptide 1 and a cDNA-mRNA dimer encoding an amino acid sequence of Peptide 1 to PCSK9.

FIG. 13 shows a graph of collection rates of binding Peptide 2 (WT) and Peptide 3 (C17S), in which C17 (F₅Ph is the first amino acid) of Peptide 2 was replaced with serine, to PCSK9.

FIG. 14 shows a MALDI-TOF mass spectrometer chart of an analysis of the cyclic peptide with FNO₂Ph having an amino acid sequence of SEQ ID: 4 (hereafter referred to as Peptide 1-1). The C-terminus of the peptide has a spacer composed of three aminohexanoic acids (Ahx) and a C-terminal biotinylated lysine.

FIG. 15 shows a MALDI-TOF mass spectrometer chart of an analysis of the cyclic peptide with FNO₂Ph having an amino acid sequence of SEQ ID: 14 (hereafter referred to as Peptide 2-1). The C-terminus of the peptide has a spacer composed of three aminohexanoic acids (Ahx) and a C-terminal biotinylated lysine.

FIG. 16 shows a chemiluminescence result of the peptide of HRP-labeled Peptide 1-1 pulled down with PCSK9-immobilized beads.

FIG. 17 shows a chemiluminescence result of the peptide of HRP-labeled Peptide 2-1 pulled down with PCSK9-immobilized beads.

FIG. 18 shows a chemiluminescence result of His-tagged PCSK9 pulled down with Peptide 1-1 bound to streptavidin beads.

FIG. 19 shows a chemiluminescence result of His-tagged PCSK9 pulled down with peptide 2-1 bound to streptavidin beads.

FIG. 20 shows a MALDI-TOF mass spectrometer chart for an analysis of dimerized Peptide 1-1.

FIG. 21 shows a chemiluminescence result of HRP-labeled Peptide 1-1 (monomer) and Peptide 1-1 dimer (Peptide A) pulled down with PCSK9-immobilized beads.

FIG. 22 shows a sequence of the collected cyclic peptide with FNO₂Ph.

FIG. 23 shows a MALDI-TOF mass spectrometer chart of an analysis of cyclic peptide with FNO₂Ph having an amino acid sequence (SEQ ID: 35) with methionine (M) replaced with isoleucine (I) in the amino acid sequence of SEQ ID: 28 (Peptide C).

FIG. 24 shows a MALDI-TOF mass spectrometer chart of an analysis of Peptide C dimer.

FIG. 25 shows a chemiluminescence result of HRP-labeled Peptide C monomer and Peptide C dimer pulled down with PCSK9-immobilized beads.

FIG. 26 shows a scheme of screening human IL-5.

FIG. 27 shows a sequence of the collected cyclic peptide with FNO₂Ph.

FIG. 28 shows a sequence of the collected cyclic peptide with F₅Ph.

FIG. 29 shows a graph of the collection rate of binding cDNA/mRNA presenting cyclic peptide with FNO₂Ph having an amino acid sequence of SEQ ID: 66 (Peptide 4) to IL-5.

FIG. 30 shows a graph of the collection rate of binding cDNA/mRNA presenting cyclic peptide with F₅Ph having an amino acid sequence of SEQ ID: 69 (Peptide 5) to IL-5.

### DESCRIPTION OF EMBODIMENTS

### Definition

For convenience, certain terms employed in the context of the present disclosure are collected here. Unless defined otherwise, all technical and scientific terms used herein have the same meaning as commonly understood by one of the ordinary skilled in the art to which this invention belongs. The singular forms “a”, “an”, and “the” are used herein to include plural referents unless the context clearly dictates otherwise.

Notwithstanding that the numerical ranges and parameters setting forth the broad scope of the invention are approximations, the numerical values set forth in the specific examples are described as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art.

Hereinafter, embodiments of the present invention are illustrated in detail. The following embodiments are illustrative only and do not limit the scope of the present invention. In order to avoid redundancy, explanation for similar contents is not repeated.

### EMBODIMENTS

Peptide Compound with Cyclic Moiety

The peptide compound of this embodiment has a cyclic moiety. The cyclic moiety has a benzoic acid derivative linker and a peptide backbone.

### Benzoic Acid Derivative Linker

A benzoic acid derivative linker is a linker derived from a benzoic acid derivative, including fluorobenzoic acid derivatives. In one embodiment, the benzoic acid derivative linker may be a benzoic acid derivative linker cyclized by an aromatic nucleophilic substitution reaction. In one embodiment, the benzoic acid derivative linker may be a compound represented by the following chemical formula (I).

### [Chemistry 15]

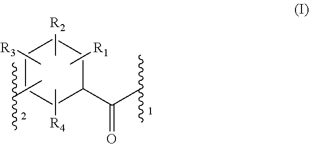

In the chemical formula (I),
R₁, R₂, R₃ and R₄ are independently —F, —H, —Cl, —Br, —SO₂NH₂, —CF₃, —SO₂CH₃ or —NO₂, respectively,
a wave line 1 represents a covalent bond with the N-terminal amino acid residue of the peptide backbone, and
a wave line 2 represents a covalent bond with the thiol group-containing residue in the peptide backbone.

In another embodiment, the benzoic acid derivative linker may be a compound represented by the following chemical formula (II) or (III).

### [Chemistry 16]

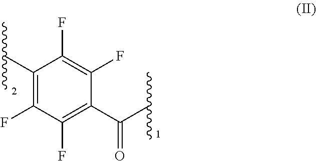 [SMILES: *C(=O)c1c(F)c(F)c(*)c(F)c1F]

### [Chemistry 17]

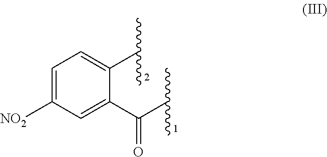 [SMILES: *C(=O)c1cc([N+](=O)[O-])ccc1*]

The benzoic acid derivative includes 2-fluoro-5-nitrobenzoic acid (FNO₂Ph) and pentafluorobenzoic acid (F₅Ph).

### Peptide Backbone

The peptide backbone has a thiol group-containing residue. The benzoic acid derivative linker binds to an N-terminal amino acid residue of the peptide backbone and a thiol group-containing residue. In one embodiment, the thiol group-containing residue is a cysteine residue. In this application, the thiol group-containing residue that is bound to the benzoic acid derivative linker can also be referred to as a linker-bound thiol-containing residue (or a linker-bound cysteine residue if the thiol group-containing residue is the cysteine residue).

The peptide backbone can contain any number of amino acid residues, e.g., 2 to 15 amino acid residues (e.g., 2, 3, 4, 5, 6, 7, 8, 9, 10, 11, 12, 13, 14 and 15 amino acid residues or a range between two values selected from these values).

The peptide backbone may contain two or more thiol-containing residues (also referred to as non-linker-bound thiol-containing residues) other than the linker-bound thiol-containing residue. In one embodiment, the N-terminal amino acid residue of the peptide backbone is an amino acid residue other than the thiol-containing residue. In another embodiment, the N-terminal amino acid residue of the peptide backbone is an amino acid residue other than the linker-bound thiol-containing residue.

When the linker-bound thiol-containing residue is located at the C-terminus of the backbone peptide, the peptide compound is cyclic. When the linker-bound thiol-containing residue is located outside the C-terminus of the backbone peptide, the peptide compound has a cyclic moiety and a linear moiety. The peptide backbone in the cyclic moiety may be referred to as a cyclic peptide backbone. The peptide backbone in the linear moiety may be referred to as a linear peptide backbone. The linear peptide backbone contains one or more amino acids.

When the linker-bound thiol-containing residue is the linker-bound cysteine residue, the peptide backbone may contain two or more cysteine residues (also referred to as non-linker-bound cysteine residues) in addition to the linker-bound cysteine residue. In one embodiment, the N-terminal amino acid residue of the peptide backbone is an amino acid residue other than a cysteine residue. In another embodiment, the N-terminal amino acid residue of the peptide backbone is an amino acid residue other than the linker-bound cysteine residue.

When the linker-bound cysteine residue is located at the C-terminus of the backbone peptide, the peptide compound is cyclic. When the linker-bound cysteine residue is located outside the C-terminus of the backbone peptide, the peptide compound has a cyclic moiety and a linear moiety. The peptide backbone in the cyclic moiety may be referred to as a cyclic peptide backbone. The peptide backbone in the linear moiety may be referred to as a linear peptide backbone. The linear peptide backbone contains one or more amino acids.

In one embodiment, the peptide compound having the cyclic moiety is a monomer. In another embodiment, the peptide compound having the cyclic moiety is a dimer composed of two peptide compounds joined together.

### Pharmaceutical Composition

The peptide compound of the embodiment may be provided as a pharmaceutical composition. The pharmaceutical composition may contain pH adjusters, buffers, stabilizers, solubilizers, and the like.

The pharmaceutical composition is administered in an effective amount to treat or prevent target diseases, whether administered alone or in combination with other therapeutic agents. However, the total amount of the peptide compound is determined by the physician in charge, within general medical judgment. The effective amount for a subject depends on the severity of the subject: the subject's age, weight, general health, sex and diet: the time of administration: the route of administration; the duration of treatment; and any drugs being used in combination or concurrently with the pharmaceutical composition. The dosage of the pharmaceutical composition need not be a constant amount for each administration. For example, it may be administered at the dosage lower than that required to achieve the desired effect and the dosage may be gradually increased until the desired effect is achieved.

If necessary, the effective daily dosage may be divided into multiple dosages depending on the purpose of administration. A person skilled in the art will be able to easily optimize the effective dosage and the combined dosage formulation depending on good medical practice and the clinical presentation of the individual subject.

The dosage of the pharmaceutical composition varies depending on conditions such as the target diseases, the subject of administration, symptoms and route of administration. For oral administration, the dosage of the pharmaceutical composition is generally about 0.01 to 1000 mg, preferably about 0.1 to 100 mg, more preferably about 0.5 to 50 mg per day in a human weighing, for example, about 60 kg. For parenteral administration, the single dose depends on conditions such as the target diseases, patient condition, symptoms and method of administration. For injectable formulations, the single dose is usually about 0.01 to 100 mg, preferably about 0.01 to 50 mg, more preferably about 0.01 to 20 mg per kilogram of body weight, for example, administered intravenously. The total dose per day may be a single dose or divided doses.

The dosage forms include intravenous, intraperitoneal, subcutaneous, intramuscular, topical, oral, parenteral, intranasal or intradermal administration. Preferably, the pharmaceutical composition is administered continuously over a period of time, i.e., 3 days or more, preferably one week or more, more preferably two weeks or more, more preferably one month or more, e.g., six months or 1 year or more. The pharmaceutical composition may be administered daily, but need not be administered daily as long as it is administered continuously over a period of time. The pharmaceutical composition may be administered in a daily dose once a day or several doses per day. Administration of the pharmaceutical composition may be terminated at the discretion of a physician or at the subject's own discretion.

In one embodiment, the peptide backbone has the amino acid sequence described in any of SEQ IDs 4 to 35 and 66 to 77. The pharmaceutical composition can treat or prevent a disease caused by a predetermined compound by binding the peptide compound to the predetermined compound. The predetermined compound may be a bioactive protein (e.g., PCSK9 (protein convertase subtilisin kexin 9)) or a cytokine (e.g., interleukin (IL) (such as IL-5), interferon (IFN), tumor necrosis factor (TNF), and chemokines). The disease may be hypercholesterolemia or allergic diseases.

tRNA

An initiator tRNA according to the embodiment has a fluorobenzoic acid derivative linker precursor represented by the following chemical formula (IV).

### [Chemistry 18]

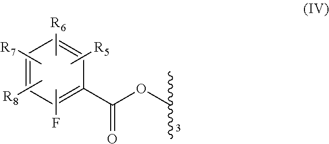

In the chemical formula (IV),
R₅, R₆, R₇ and R₅ are independently —F, —H, —Cl, —Br, —SO₂NH₂, —CF₃, —SO₂CH₃ or —NO₂, respectively, and
a wave line 3 represents a covalent bond with tRNA.

The initiator tRNA with a fluorobenzoate derivative linker precursor are paired with mRNA initiation codons (e.g., AUG) as an alternative compound to aminoacyl-tRNA during translation of mRNA in the ribosome. After the initiator tRNA pairs with the initiation codon of the mRNA, amino acids are sequentially bound to the fluorobenzoic acid derivative linker precursor as a starting point. During or after the binding process of the precursor and the amino acid, the fluorobenzoate derivative linker precursor and the peptide spontaneously cyclize. The cyclization results in the formation of a compound with a cyclic moiety. By the cyclization, the fluorobenzoic acid derivative linker precursor is present as a benzoic acid derivative linker.

The fluorobenzoic acid derivative linker precursor may be a compound represented by the following chemical formula (V) or (VI):

### [Chemistry 19]

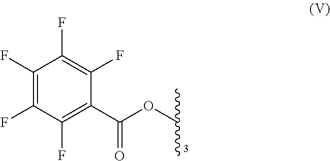 [SMILES: *OC(=O)c1c(F)c(F)c(F)c(F)c1F]

### [Chemistry 20]

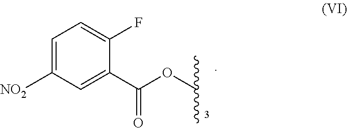 [SMILES: *OC(=O)c1cc([N+](=O)[O-])ccc1F]

### Method for Producing the Peptide Compound

The method for producing the peptide compounds according to the embodiment includes:
a providing step for providing at least one type of mRNA;
a translation step for translating the mRNA in a presence of the initiator tRNA; and
the mRNA has an upstream nucleotide sequence having a nucleotide sequence corresponding to a start codon and a downstream nucleotide sequence having a nucleotide sequence encoding the peptide backbone.

The upstream nucleotide sequence has a nucleotide sequence corresponding to the start codon to which the initiator tRNA with the fluorobenzoic acid derivative linker precursor is paired. The nucleotide sequence corresponding to the initiation codon is usually an AUG sequence, but may be a nucleotide sequence other than the AUG sequence (e.g., AUA and GUG) depending on the producing method and producingconditions. The nucleotide sequence corresponding to the start codon is located at the 3′ end of the upstream nucleotide sequence. In one embodiment, the upstream nucleotide sequence has a nucleotide sequence of the 5′ end untranslated region (UTR) and a nucleotide sequence corresponding to the start codon located at the 3′ end thereof.

The downstream nucleotide sequence has a nucleotide sequence encoding the peptide backbone. The downstream nucleotide sequence is linked to the 3′ end of the upstream nucleotide sequence. In one embodiment, the downstream nucleotide sequence has the nucleotide sequence encoding the peptide backbone and the 3′ end untranslated region at 3′ end thereof. The nucleotide sequence of the 5′ end untranslated region (UTR) in the upstream nucleotide sequence and the nucleotide sequence of the 3′ end untranslated region in the downstream nucleotide sequence may be identical or different from each other.

Translation of the mRNA takes place in the presence of the initiator tRNA. This causes the fluorobenzoic acid derivative linker precursor to bind to the peptide backbone. In one embodiment, translation of the mRNA is performed in a cell-free protein synthesis system (e.g., PURE system) in the presence of the initiator tRNA. Components used in the PURE system can include ribosomes, T7 RNA polymerase, IF-1, IF-2, IF-3, EF-Tu, EF-Ts, EF-G, RRF, AlaRS, CysRS, AspRS, GluRS, PheRS, GlyRS, HisRS, IleRS, LysRS, LeuRS, MetRS, AsnRS, ProRS, GlnRS, ArgRS, SerRS, ThrRS, ValRS, TrpRS, TyrRS, NDK, myo kinase, creatine kinase, E. coli total tRNA, Hepes buffer, potassium acetate, magnesium acetate, ATP, GTP, UTP, CTP, creatine phosphate, DTT, spermidine.

The at least one type of mRNA may be two or more types of mRNAs. If the at least one type mRNA is two or more types of mRNAs, each of the nucleotide sequences encoding the peptide backbone may be based on nucleotide sequences created so as to have a random amine acid sequence.

The peptide compound can also be chemically synthesized by Fmoc solid-phase peptide synthesis.

The method for producing the peptide compound according to this embodiment may further include:
a DNA providing step for providing DNA having an upstream nucleotide sequence having a nucleotide sequence corresponding to a start codon and a downstream nucleotide sequence encoding the peptide backbone; and
a transcription step for perform transcription of the DNA.

### Method for Screening Target Protein

A method for screening a target protein according to the embodiment, including:
a providing step for providing at least one type of mRNA;
a translation step for translating the mRNA in a presence of the initiator tRNA and a puromycin DNA linker to obtain a conjugate of a peptide compound having a cyclic moiety with a nucleic acid;
a contact step for contacting the conjugate with the target protein; and
an analysis step for analyzing a binding of the target protein and the conjugate;
in which the mRNA has an upstream nucleotide sequence having a nucleotide sequence corresponding to a start codon and a downstream nucleotide sequence having a nucleotide sequence encoding the peptide backbone.

The puromycin DNA linker has DNA with a nucleotide sequence complementary to the 3′ end untranslated region of mRNA and puromycin bound to the DNA. When a puromycin-modified mRNA is translated in the ribosome, the puromycin is added to the C-terminus of the translated peptide backbone, while the mRNA remains bound to the puromycin.

In the conjugate of the peptide compound having the cyclic moiety with a nucleic acid, the peptide compound having the cyclic moiety is bound to the nucleic acid via the puromycin. In one embodiment, the nucleic acid is mRNA. In another embodiment, the nucleic acid is double-stranded mRNA/cDNA. The nucleic acid is preferably the double-stranded mRNA/cDNA that is highly resistant to degradation. The method for screening the target protein may further include a reverse transcription step for perform reverse transcription of the mRNA in the conjugate to the double-stranded mRNA/cDNA.

The target protein may have a substance (e.g., His tag) that facilitates collection.

The analysis step for analyzing the binding of the target protein and the conjugate may further include a collection step for collecting the target protein or the conjugate. Whether to collect the target protein or the conjugate may be determined by the difficulty level of collection. The analysis step for analyzing the binding of the target protein and the conjugate may further include a detection step for detecting the target protein. The target protein can be detected by chemiluminescence, for example, by using a fluorescent label. The analysis step for analyzing the binding of the target protein and the conjugate may further include a determination step for determining the nucleotide sequence of the nucleic acid in the conjugate.

The method for screening the target protein according to this embodiment may further include a repeat step in which a series of steps including the translation step, the contact step, and the analysis step are repeated one or more times using mRNA having the nucleotide sequence determined by the determination step for determining the nucleotide sequence of the nucleic acid in the conjugate.

The present invention includes each of the following inventions.

A method for preventing or treating the target disease, including administering to a mammal an effective amount of the peptide compound having the cyclic moiety or a prodrug thereof.

Use of the peptide compound having the cyclic moiety or the prodrug thereof to producing prophylactic or therapeutic agents for the target disease.

A peptide library containing the peptide compounds having various amino acid sequences, wherein the peptide compounds are peptide compounds having cyclic moieties.

### EXAMPLES

### Experimental Example 1

### Peptide Compound Having Cyclic Moiety

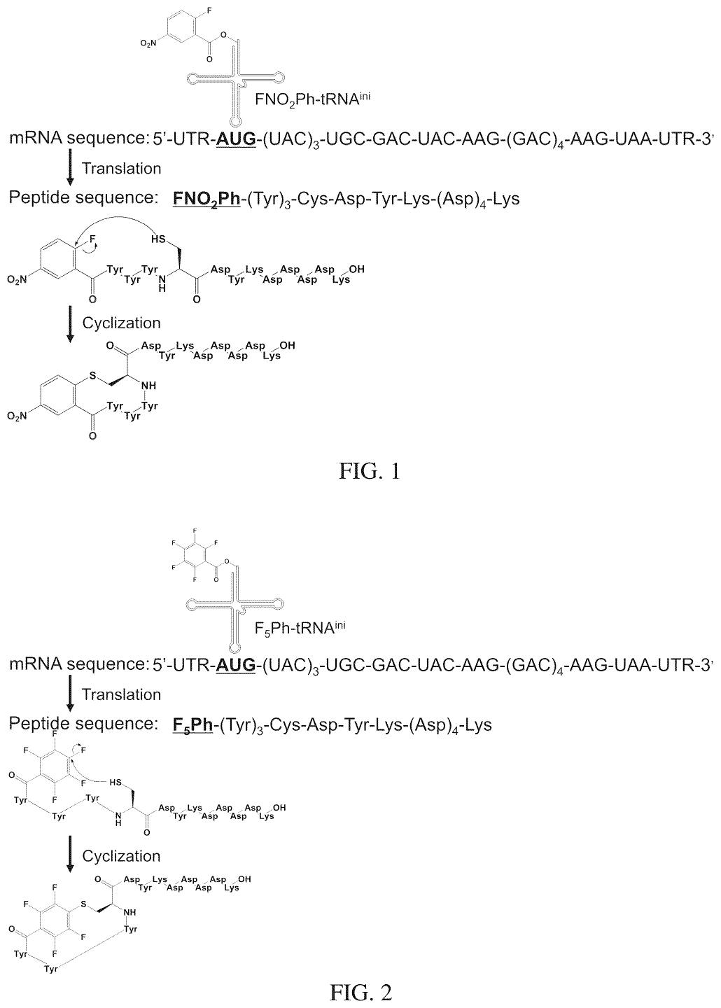

A cyclic peptide with 2-fluoro-5-nitrobenzoic acid (FNO₂Ph) as a benzoic acid derivative linker and a cyclic peptide with and pentafluorobenzoic acid (F₅Ph) as a benzoic acid derivative linker were produced. FIGS. 1 and 2 show the production schemes for the cyclic peptides with FNO₂Ph and F₅Ph, respectively. Each cyclic peptide was produced using the PURE system (NEB, PURExpress). Components used in the PURE system are ribosomes, T7 RNA polymerase, IF-1, IF-2, IF-3, EF-Tu, EF-Ts, EF-G, RRF, AlaRS, CysRS, AspRS, GluRS, PheRS, GlyRS, HisRS, IleRS, LysRS, LeuRS, MetRS, AsnRS, ProRS, GlnRS, ArgRS, SerRS, ThrRS, ValRS, TrpRS, TyrRS, NDK, myo kinase, creatine kinase, E. coli total tRNA, Hepes buffer, potassium acetate, magnesium acetate, ATP, GTP, UTP, CTP, creatine phosphate, DTT, and spermidine.

E. coli initiator tRNA (tRNAⁱⁿⁱ) was prepared by in vitro transcription of the appropriate DNA template using T7 RNA Polymerase (RNAP) and purified by ethanol precipitation. 2-Fluoro-5-nitrobenzoic acid (FNO₂Ph) with acyl-tRNAⁱⁿⁱ and pentafluorobenzoic acid (F₅Ph) with acyl-tRNAⁱⁿⁱ were prepared using N-cyclohexyl-2-aminoethanesulfonate buffer.

The prepared FNO₂Ph-tRNAᵢₙᵢ and F₅Ph-tRNAᵢₙᵢ were purified by ethanol precipitation. Template DNA (SEQ ID: 2) encoding the model fMet-(Tyr) 3-Cys-Asp-Tyr-Lys-(Asp) 4-Lys peptide (SEQ ID: 1) was amplified by PCR. The template DNA was transcribed and translated using the PURE system containing four protein-generating amino acids (Cys, Asp, Tyr, Lys) and FNO₂Ph-tRNAᵢₙᵢ or F₅Ph-tRNAᵢₙᵢ for 1 hour at 37° C. Translation products were incubated with reverse transcription buffer at pH 8.4 for 30 min at 42° C. Expressed peptides were desalted and analyzed using a MALDI-TOF (Matrix Assisted Laser Desorption/Ionization-Time-Flight) mass spectrometer (MS).

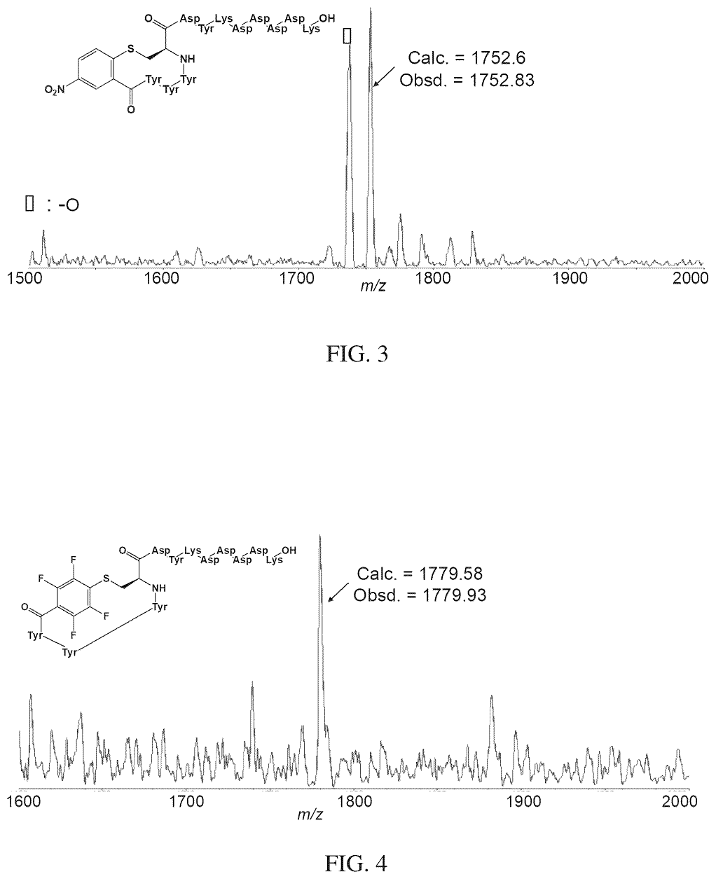

As shown in FIGS. 3 and 4, each linker was incorporated into the respective peptides in the ribosome, indicating that each peptide spontaneously underwent aryl-thioether cyclization.

### Experimental Example 2

Screening for Peptides that Bind to Human PCSK9

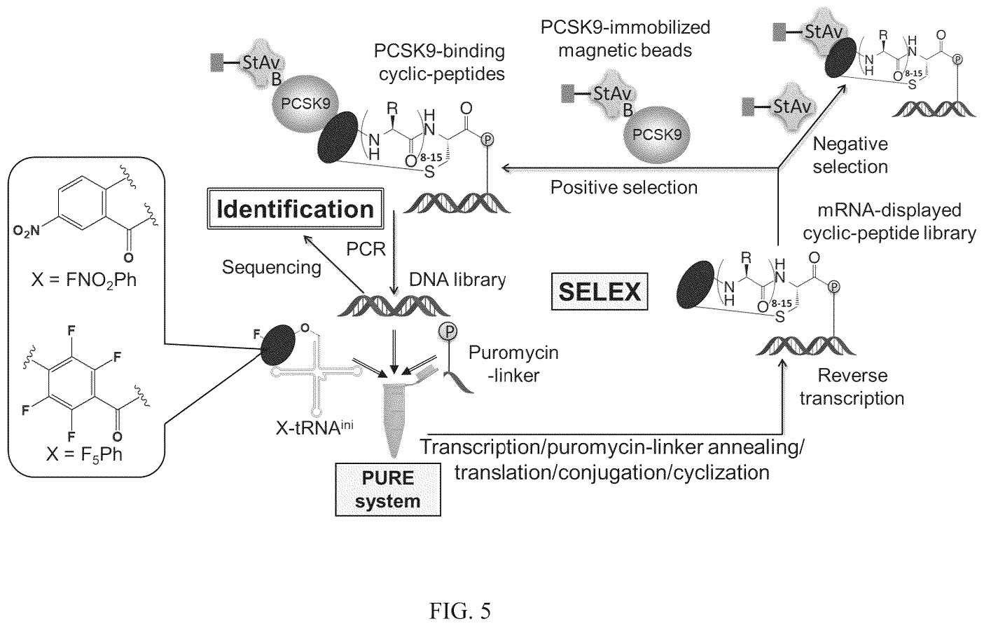

A screening was performed on peptides that bind to human PCSK9. FIG. 5 shows the scheme of the screening.

Site-specific biotinylated human PCSK9 was immobilized on streptavidin-modified magnetic beads. Fc fusion protein of human PCSK9 was immobilized on protein A-modified magnetic beads. Immobilization of PCSK9 was confirmed by sodium dodecyl sulfate polyacrylamide gel electrophoresis and staining with Coomassie brilliant blue.

Eight template DNA libraries encoding 8 to 15 random amino acids between the cyclic compound (FNO₂Ph or F₅Ph) and the downstream cysteine residue were prepared by PCR amplification of the template DNA prepared by primer extension. The prepared template DNA libraries were transcribed into mRNA libraries by run-off in vitro transcription using T7 RNAP.

Each Library of mRNA-presenting FNO₂Ph and F₅Ph cyclized peptides was prepared by in vitro translation at 37° C. for 25 min, using a termination factor (RF)-free PURE system including 0.5 mM of 19 protein-producing amino acids (excluding Met), 3 μM mixed mRNA library, 3 μM puromycin-DNA linker, and 100 μM FNO₂Ph-tRNAⁱⁿⁱ or F₅Ph-tRNAⁱⁿⁱ.

The puromycin DNA linker has a DNA sequence complementary to the 3′ end untranslated region of the mRNA (CCCGCCTCCCGCCCCCCGTCC (SEQ ID: 3)), with (Spc18)₅-CC-puromycin linked to the 3′ end of the DNA sequence (Spc18 is Spacer 18 (hexaethyleneglycol)). The puromycin DNA linker was synthesized by Bex Co. LTD. When the puromycin-modified mRNA is translated in ribosomes, the puromycin is added to the C-terminus of the translated peptide, while the mRNA remains bound to the puromycin. The acquired peptide spontaneously cyclizes.

EDTA was added to each of the mRNA-presenting FNO₂Ph and F₅Ph cyclized peptide libraries. The mRNA-presenting peptide libraries were reverse transcribed to form double-stranded mRNA/cDNA using RNase H-inactivated reverse transcriptase at 42° C. for 30 minutes. The reverse transcription was quenched with EDTA and neutralized with HEPES.

Each of the mRNA-presenting FNO₂Ph and F₅Ph cyclized peptide libraries was incubated with PCSK9-immobilized streptavidin-modified beads as positive selection. After removal of the supernatant, the beads were washed with HBS-T. cDNA encoding the peptide binding to the PCSK9-immobilized beads was PCR amplified with Taq DNA polymerase. The amplified PCR product was used for the next round of SELEX.

In the next round, in vitro transcription and translation were performed in an RF-free PURE system containing 19 protein-producing amino acids (excluding Met), puromycin-DNA linker, FNO₂Ph-tRNAⁱⁿⁱ or F₅Ph-tRNAⁱⁿⁱ, and the DNA library obtained in the previous round to prepare mRNA-presenting peptide libraries. The mRNA-presenting peptide libraries were reverse transcribed and the reverse transcription products were mixed with streptavidin-modified beads and the supernatant was removed from the mixture. This process, referred to as negative selection, was performed five times to remove peptides bound to the streptavidin-modified beads.

After the positive selection, cDNA encoding peptides that bind to PCSK9 immobilized beads was amplified by PCR and quantified by qPCR using SYBR green.

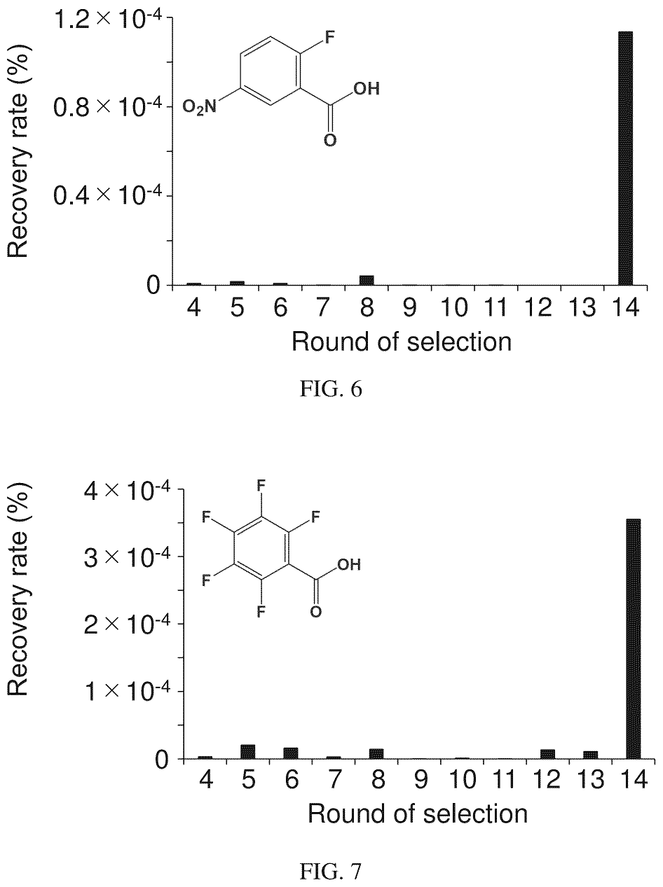

The above-mentioned round was performed 14 times. FIG. 6 shows the collection rate of cDNA in each round of this scheme by FNO₂Ph cyclized peptide. FIG. 7 shows the collection rate of cDNA in each round of this scheme by F₅Ph cyclized peptides. Both cyclic peptides showed a significant increase in the collection rate in round 14.

After the final round of SELEX, each library was sequenced by the Sanger method or next-generation sequencing. Template focus DNA libraries for in vitro affinity maturation were prepared by PCR amplification of the template DNA prepared by primer extension. The template focus DNA library was transcribed into a focus mRNA library by in vitro transcription using T7 RNAP.

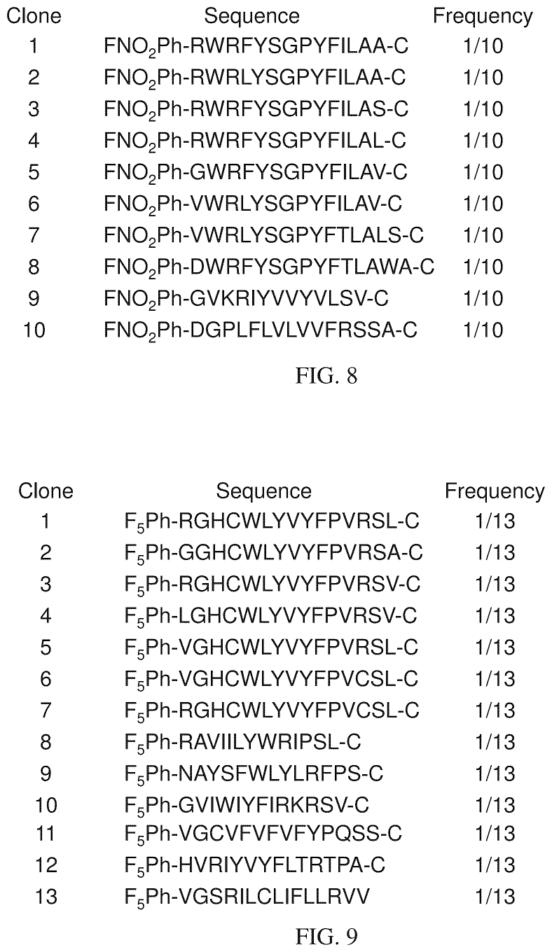

FIG. 8 shows the sequence of the FNO₂Ph cyclized peptide. FIG. 9 shows the sequence of the F₅Ph cyclized peptide. Table 1 shows each SEQ ID corresponding to the amino acid sequence of each peptide.

TABLE 1

| | | | | SEQ ID |
|---|---|---|---|---|
| | | | | of the |
| | | | | corresponding |
| Clone | | amino acid | SEQ | nucleotide |
| No. | Linker | sequence | ID | sequence |
| 1 | FNO₂Ph | RWRFYSGPYFILAAC | 4 | 36 |
| 2 | FNO₂Ph | RWRLYSGPYFILAAC | 5 | 37 |
| 3 | FNO₂Ph | RWRFYSGPYFILASC | 6 | 38 |
| 4 | FNO₂Ph | RWRFYSGPYFILALC | 7 | 39 |
| 5 | FNO₂Ph | GWRFYSGPYFILAVC | 8 | 40 |
| 6 | FNO₂Ph | VWRLYSGPYFILAVC | 9 | 41 |
| 7 | FNO₂Ph | VWRLYSGPYFTLALSC | 10 | 42 |
| 8 | FNO₂Ph | DWRFYSGPYFTLAWAC | 11 | 43 |
| 9 | FNO₂Ph | GVKRIYVVYVLSVC | 12 | 44 |
| 10 | FNO₂Ph | DGPLFLVLVVFRSSAC | 13 | 45 |
| 1 | F₅Ph | RGHCWLYVYFPVRSLC | 14 | 46 |
| 2 | F₅Ph | GGHCWLYVYFPVRSAC | 15 | 47 |
| 3 | F₅Ph | RGHCWLYVYFPVRSVC | 16 | 48 |
| 4 | F₅Ph | LGHCWLYVYFPVRSVC | 17 | 49 |
| 5 | F₅Ph | VGHCWLYVYFPVRSLC | 18 | 50 |
| 6 | F₅Ph | VGHCWLYVYFPVCSLC | 19 | 51 |
| 7 | F₅Ph | RGHCWLYVYFPVCSLC | 20 | 52 |
| 8 | F₅Ph | RAVIILYWRIPSLC | 21 | 53 |
| 9 | F₅Ph | NAYSFWLYLRFPSC | 22 | 54 |
| 10 | F₅Ph | GVIWIYFIRKRSVC | 23 | 55 |
| 11 | F₅Ph | VGCVFVFVFYPQSSC | 24 | 56 |
| 12 | F₅Ph | HVRIYVYFLTRTPAC | 25 | 57 |
| 13 | F₅Ph | VGSRILCLIFLLRVV | 26 | 58 |

### Experimental Example 3

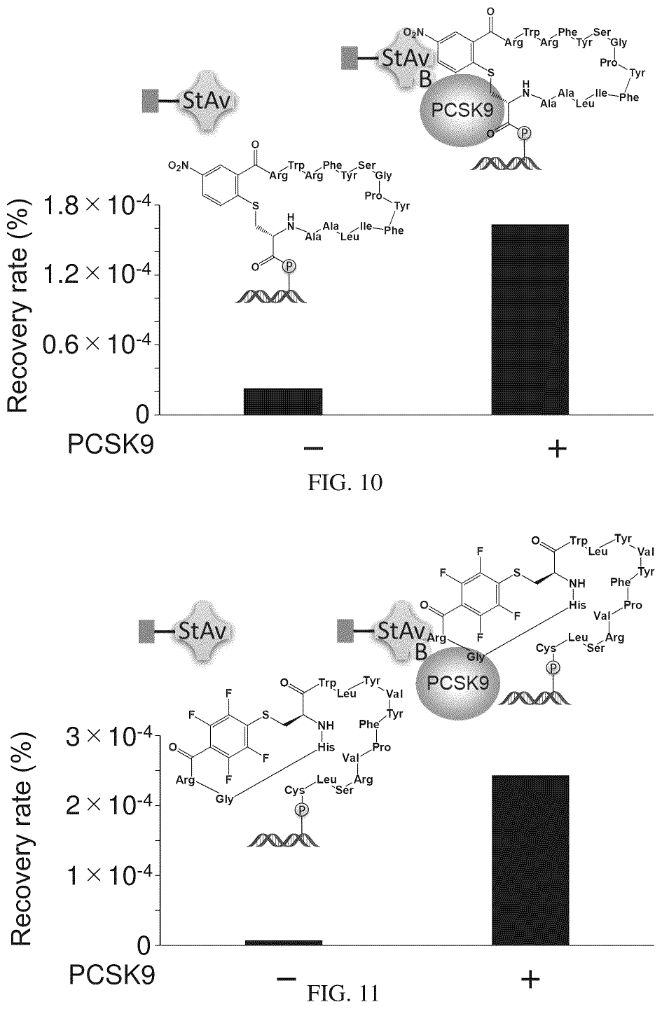

Each collection rate of the cDNA/mRNA presenting FNO₂Ph cyclized peptide with the amino acid sequence of SEQ ID: 4 (hereafter referred to as Peptide 1) and the cDNA/mRNA presenting F₅Ph cyclized peptide with the amino acid sequence of SEQ ID: 14 (hereafter referred to as Peptide 2) against PCSK9 was determined. PCSK9-immobilized beads were used as negative controls. Each collection rate of PCSK9 for Peptides 1 and 2 was higher than that of the negative control (FIGS. 10 and 11).

### Experimental Example 4

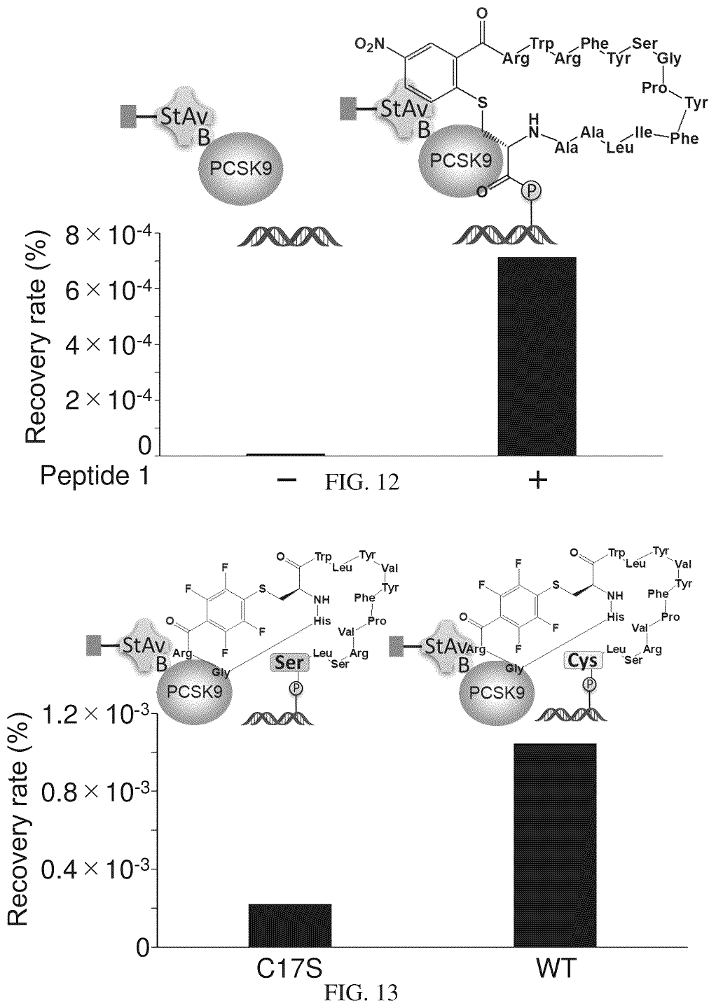

Each collection rate of Peptide 1 and the cDNA-mRNA dimer encoding the amino acid sequence of Peptide 1 to PCSK9 was measured (FIG. 12), revealing that the binding of Peptide 1 to PCSK9 was peptide-dependent.

### Experimental Example 5

Peptide 3 (SEQ ID: 27) was prepared by replacing C17 in Peptide 2 (F₅Ph is the first amino acid) with serine. Each collection rate of Peptide 2 (WT) and Peptide 3 (C17S) against PCSK9 was measured (FIG. 13). C17 in Peptide 2 was found to be critical for Peptide 2 binding to PCSK9.

### Experimental Example 6

FNO₂Ph cyclized peptide with the amino acid sequence according to SEQ ID: 4 (hereafter referred to as Peptide 1-1) and FNO₂Ph cyclized peptide with the amino acid sequence according to SEQ ID: 14 (hereafter referred to as Peptide 2-1) were chemically synthesized. The C-terminus of each peptide has a spacer composed of three aminohexanoic acids (Ahx) and a C-terminal biotinylated lysine.

Each peptide was synthesized using a standard solid-phase peptide synthesis method with 9-fluorenylmethyloxycarbonyl (Fmoc)-protected amino acids on Rink amide resin. Amino acid coupling, FNO₂Ph coupling, and F₅Ph coupling were performed using HBTU, HOBt, and DIEA. The peptides were deprotected and excised from the resin using a mixture of H₂O, triisopropylsilane, 3,6-dioxa-1,8-octanedithiol, and trifluoroacetic acid. The excised peptides were filtered from the resin, precipitated by centrifugation in diethyl ether, and dried in vacuum. Linear peptides were cyclized with triethylamine in DMF and analyzed by MALDI-TOF MS and quantified by measuring absorbance at 280 nm using a spectrophotometer.

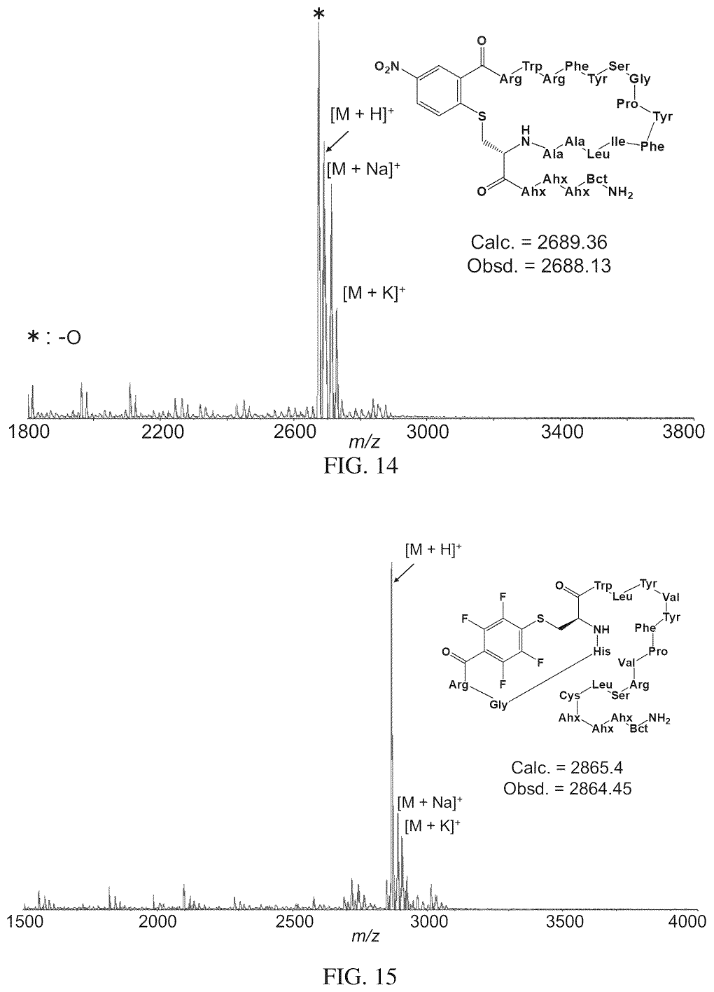

Each chemical dimer of FNO₂Ph and F₅Ph cyclized peptides was synthesized using N-α, ε-di-Fmoc-L-lysine. Amino acid coupling, FNO₂Ph coupling and F₅Ph coupling were performed using HATU, HOAt and DIEA. The peptides were deprotected, truncated, cyclized, analyzed by MALDI-TOF MS (FIGS. 14 and 15), and quantified as described above.

### Experimental Example 7

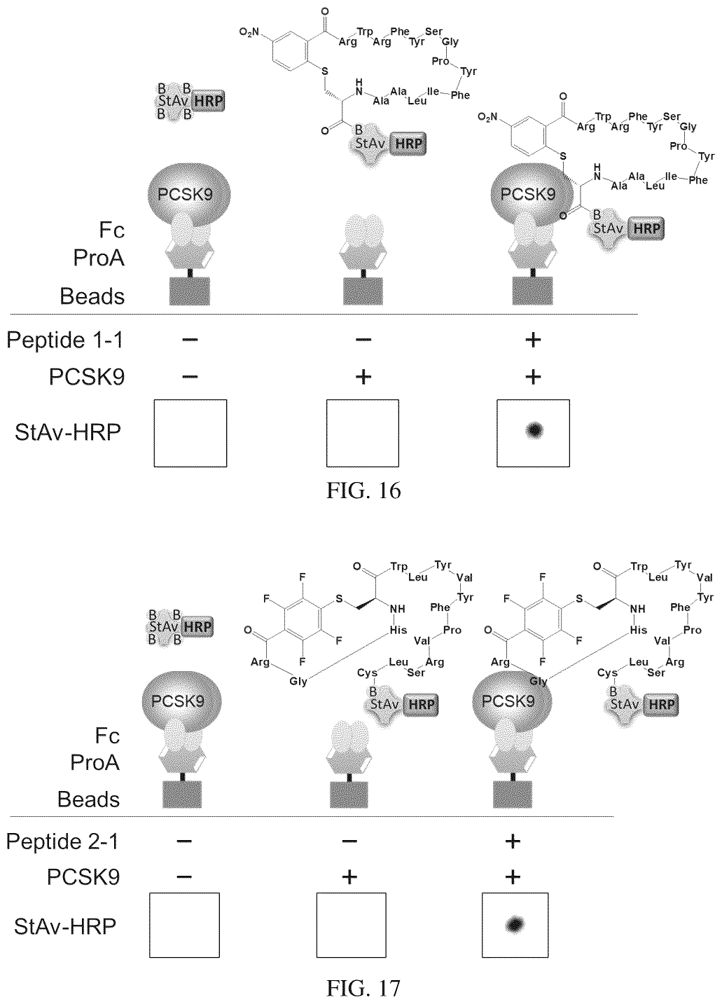

Peptide 1-1 and Peptide 2-1 were labeled via horseradish peroxidase (HRP)-labeled streptavidin. Each peptide of HRP-labeled Peptide 1-1 and Peptide 2-1 was pulled down with PCSK9-immobilized beads. HRP substrate was added to PCSK9-immobilized beads and chemiluminescence was detected using LuminoGraph III (ATTO). As shown in FIGS. 16 and 17, Peptide 1-1 and Peptide 2-1 were found to have binding activity to PCSK9 even without presentation by the cDNA-mRNA dimer.

### Experimental Example 8

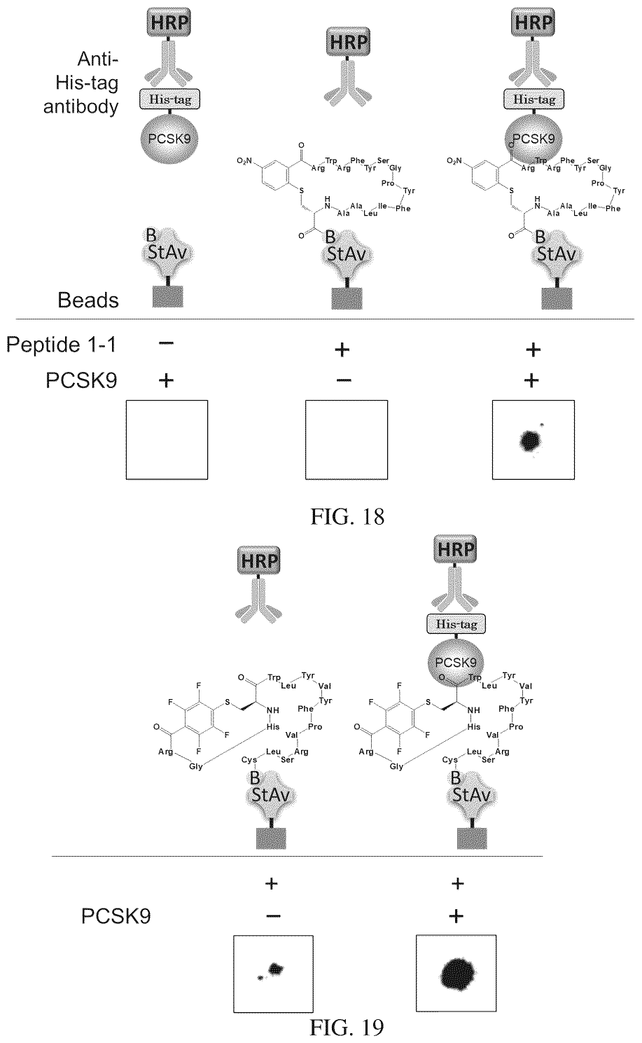

Each of Peptide 1-1 and peptide 2-1 was conjugated to streptavidin beads. His-tagged PCSK9 was labeled via HRP-conjugated anti-His-tagged antibody. The complex of PCSK9 and HRP was pulled down with Peptide 1-1 or Peptide 2-1 immobilized beads. HRP substrates were added to the peptide-immobilized beads and chemiluminescence was detected using LuminoGraph III (ATTO). As shown in FIGS. 18 and 19, Peptide 1-1 and Peptide 2-1 were found to have binding activity to PCSK9 even without presentation by the cDNA-mRNA dimer.

### Experimental Example 9

### Peptide Dimer

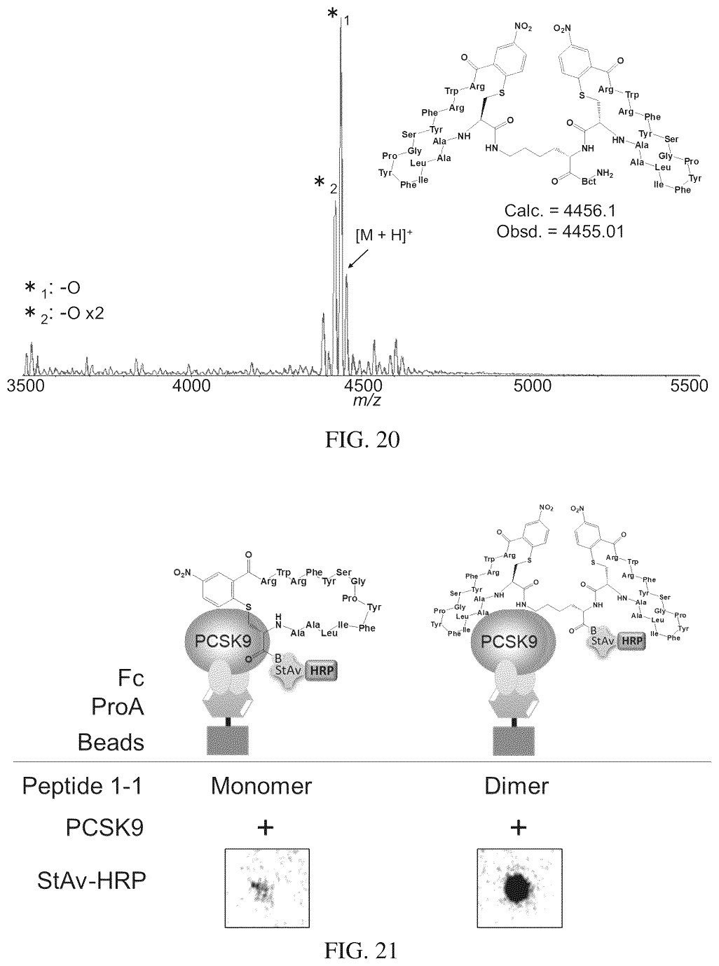

To improve affinity (avidity), Peptide 1-1 was chemically dimerized. The dimer of the FNO₂Ph cyclized peptide was chemically synthesized by Fmoc solid phase peptide synthesis using a C-terminal biotinylated lysine. The dimer of Peptide 1-1 was confirmed by MALDI-TOF MS (FIG. 20).

The PCSK9 binding activity of HRP-labeled Peptide 1-1 (monomer) and the dimer of Peptide 1-1 (Peptide A) were analyzed by PCSK9 pull-down and chemiluminescence detection. As shown in FIG. 21, the PCSK9 binding activity of the dimer of Peptide 1-1 (Peptide A) was found to be higher than that of Peptide 1-1 (monomer).

### Experimental Example 10

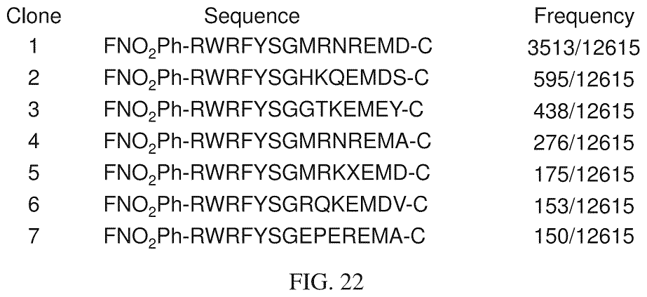

FNO₂Ph cyclized peptide was prepared by randomizing 7 amino acid residues from the N-terminus (RWRFYSG) or 7 amino acid residues composed of the second to eighth amino acid residues from the C-terminus (PYFILAA) in the amino acid sequence of SEQ ID: 4. F₅Ph cyclized peptides was also prepared by randomizing 7 amino acid residues from the N-terminus (RGHCWLY) or 7 amino acid residues from the C-terminal cysteine (YFPVRSL) in the amino acid sequence of SEQ ID: 14. The peptides that bind to PCSK9 were identified based on the same method as in Experimental Example 2. FIG. 22 shows the sequence of the FNO₂Ph cyclized peptides. Table 2 shows the SEQ IDs corresponding to the amino acid sequence of each peptide. The peptides randomized to the N-terminal 7 amino acid residues (PYFILAA) in the FNO₂Ph cyclized peptide did not converge to a specific peptide sequence.

TABLE 2

| | | | | SEQ ID |
|---|---|---|---|---|
| | | | | of the |
| | | | | corresponding |
| Clone | | Amino acid | SEQ | nucleotide |
| No. | Linker | sequence | ID | sequence |
| 1 | FNO₂Ph | RWRFYSGMRNREM | 28 | 59 |
| | | DC | | |
| 2 | FNO₂Ph | RWRFYSGHKQEMD | 29 | 60 |
| | | SC | | |
| 3 | FNO₂Ph | RWRFYSGGTKEME | 30 | 61 |
| | | YC | | |
| 4 | FNO₂Ph | RWRFYSGMRNREM | 31 | 62 |
| | | AC | | |
| 5 | FNO₂Ph | RWRFYSGMRKXEM | 32 | 63 |
| | | DC | | |
| 6 | FNO₂Ph | RWRFYSGRQKEMD | 33 | 64 |
| | | VC | | |
| 7 | FNO₂Ph | RWRFYSGEPEREM | 34 | 65 |
| | | AC | | |

“X” in the amino acid sequence indicates any amino acid. Since the translation system (PURE system) used in the experiment does not contain a translation termination factor RF, translation does not terminate at the termination codon in the nucleotide sequence, but translation to any amino acid occurs (in most cases, glutamine (Q), which is close to a sequence of the codon).

### Experimental Example 11

### Met to Ile Mutation

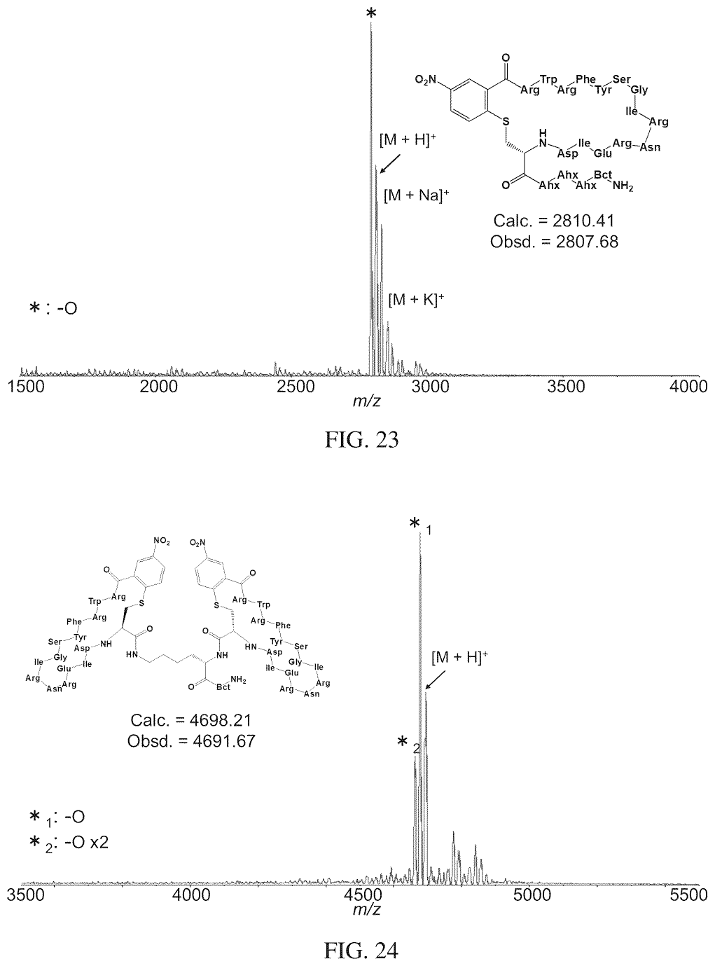

FNO₂Ph cyclized peptide (Peptide C) with an amino acid sequence (SEQ ID: 35) in which two methionines (M) in the amino acid sequence of SEQ ID: 28 were replaced with isoleucine (I) was prepared. Peptide C was chemically synthesized using the Fmoc solid-phase peptide synthesis method. The C-terminus of each peptide has a spacer composed of three aminohexanoic acids (Ahx) and a C-terminal biotinylated lysine. In addition, the dimer of Peptide C was also prepared based on the method of Experimental Example 9. Each peptide prepared was confirmed by MALDI-TOF MS (FIGS. 23 and 24).

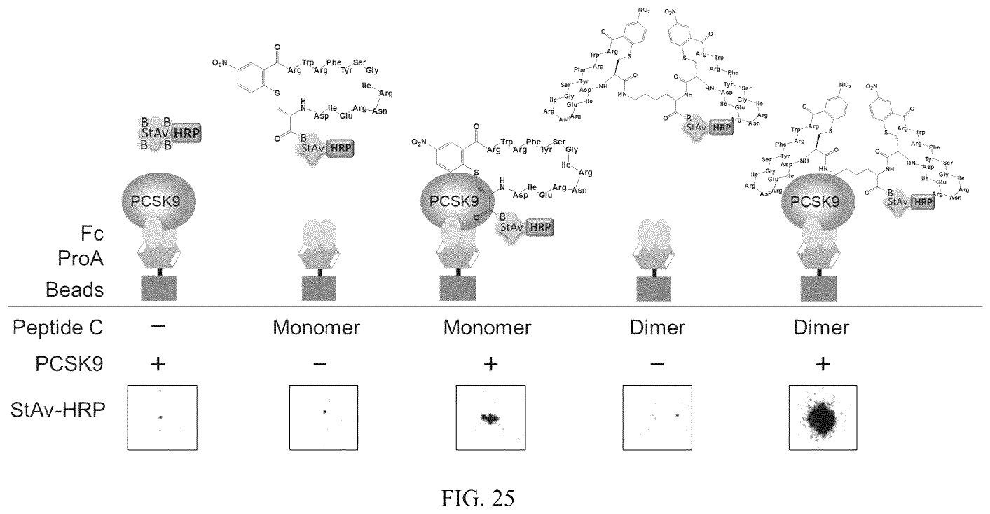

Peptide C was labeled via HRP-labeled streptavidin. Based on the same method as in Experimental Example 7, the PCSK9 binding activity of HRP-labeled Peptide C was analyzed by PCSK9 pulldown and chemiluminescence detection. As shown in FIG. 25, Peptide C was found to have binding activity for PCSK9 and the PCSK9 binding activity of the dimer of Peptide C was higher than that of the monomer of Peptide C.

### Experimental Example 12

Screening for Peptides that Bind to Human IL-5

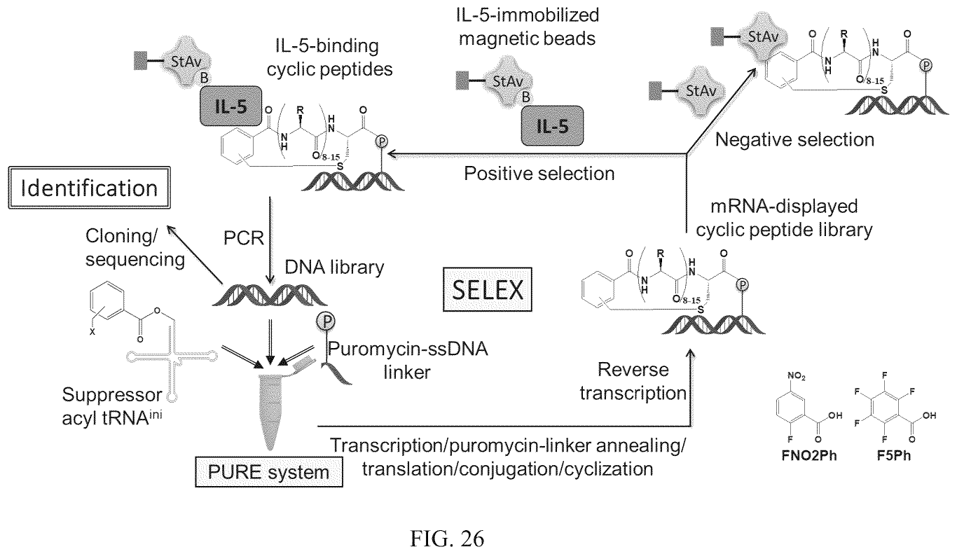

We checked whether the screening in Experimental Example 2 is applicable to compounds other than human PCSK9. Screening was performed for peptides that bind to human IL-5 as compounds other than human PCSK9. FIG. 26 shows the scheme of the screening, which was performed according to the procedure described in Experimental Example 2, except that IL-5 was used instead of PCSK9.

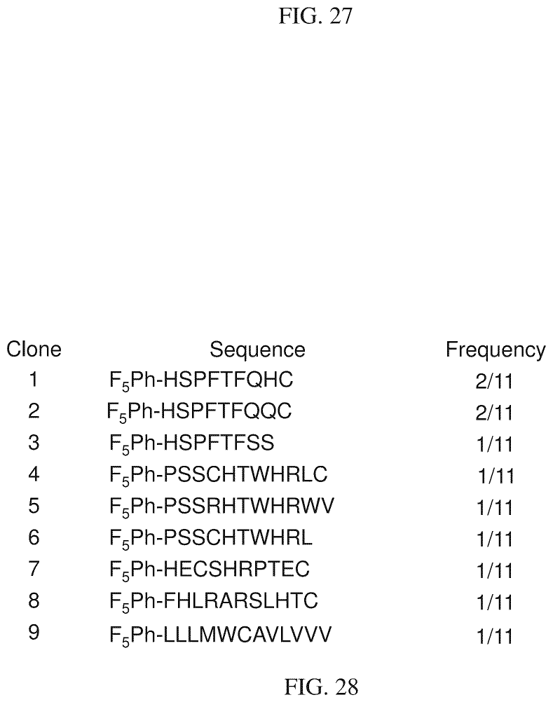

FIG. 27 shows the sequence of the FNO₂Ph cyclized peptide. FIG. 28 shows the sequence of the F₅Ph cyclized peptide. Table 3 shows the SEQ IDs corresponding to the amino acid sequence of each peptide.

TABLE 3

| | | | | SEQ ID |
|---|---|---|---|---|
| | | | | of the |
| | | | | corresponding |
| Clone | | Amino acid | SEQ | nucleotide |
| No. | Linker | sequence | ID | sequence |
| 1 | FNO₂Ph | LLLMWCAVLVVL | 66 | 78 |
| | | RWRRR | | |
| 2 | FNO₂Ph | LLLMWCAVLVVV | 67 | 79 |
| | | RWRRR | | |
| 3 | FNO₂Ph | AFLLLWSHGRPR | 68 | 80 |
| | | IIFC | | |
| 1 | F₅Ph | HSPFTFQHC | 69 | 81 |
| 2 | F₅Ph | HSPFTFQQC | 70 | 82 |
| 3 | F₅Ph | HSPFTFSS | 71 | 83 |
| 4 | F₅Ph | PSSCHTWHRLC | 72 | 84 |
| 5 | F₅Ph | PSSRHTWHRWV | 73 | 85 |
| 6 | F₅Ph | PSSCHTWHRL | 74 | 86 |
| 7 | F₅Ph | HECSHRPTEC | 75 | 87 |
| 8 | F₅Ph | FHLRARSLHTC | 76 | 88 |
| 9 | F₅Ph | LLLMWCAVLVVV | 77 | 89 |

### Experimental Example 13

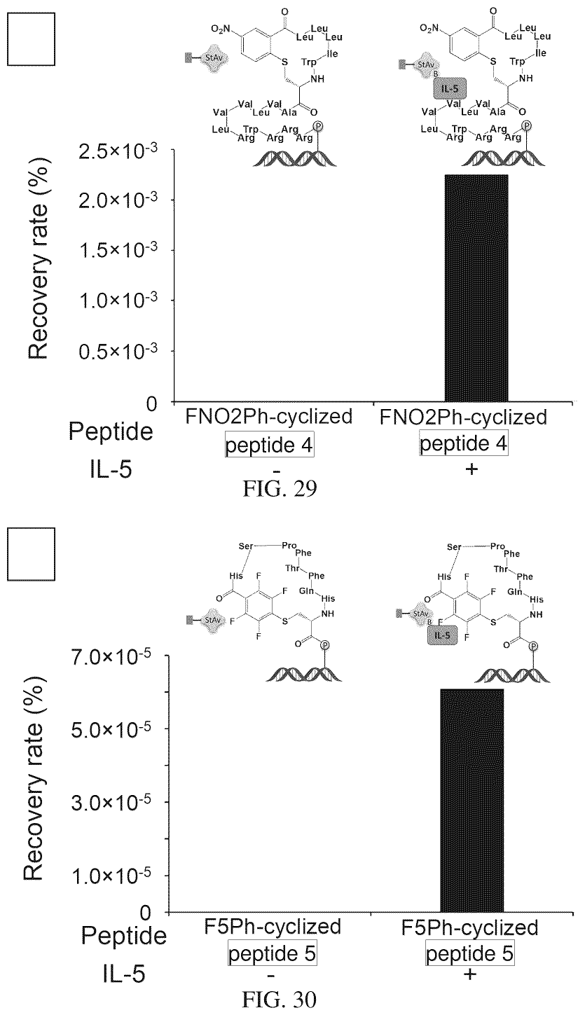

Each collection rate of cDNA/mRNA presenting FNO₂Ph cyclized peptide with the amino acid sequence of SEQ ID: 66 (hereafter referred to as Peptide 4) and cDNA/mRNA presenting F₅Ph cyclized peptide with the amino acid sequence of SEQ ID: 69 (hereafter referred to as Peptide 5) against IL-5 was determined. IL-5 non-immobilized beads were used as a negative control. The collection rates of IL-5 for Peptides 4 and 5 were higher than those of the negative control (FIGS. 29 and 30). From the above, it was possible to screen for human IL-5 which is a compound other than human PCSK9.

We claim:

1. A peptide compound having a cyclic moiety,
wherein the cyclic moiety has:
a benzoic acid derivative linker to be cyclized by an aromatic nucleophilic substitution reaction; and
a peptide backbone,
the peptide backbone has a thiol group-containing residue, and
the benzoic acid derivative linker is bound to the peptide backbone via an N-terminal amino acid residue of the peptide backbone and the thiol group-containing residue.

2. The peptide compound according to claim 1, wherein the benzoic acid derivative linker is a compound represented by the following chemical formula (I):
[Chemistry 1] 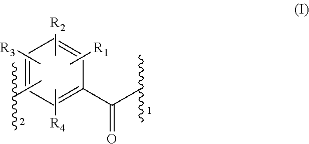
(in the chemical formula (I),
R₁, R₂, R₃ and R₄ are independently —F, —H, —Cl, —Br, —SO₂NH₂, —CF₃, —SO₂CH₃ or —NO₂, respectively,
a wave line 1 represents a covalent bond with the N-terminal amino acid residue of the peptide backbone, and
a wave line 2 represents a covalent bond with the thiol group-containing residue in the peptide backbone).

3. A peptide compound having a cyclic moiety,
wherein the cyclic moiety has:
a benzoic acid derivative; and
a peptide backbone,
the peptide backbone has a thiol group-containing residue,
the benzoic acid derivative linker is bound to the peptide backbone via an N-terminal amino acid residue of the peptide backbone and the thiol group-containing residue, and
the benzoic acid derivative linker is a compound represented by the following chemical formula (I):
[Chemistry 2] 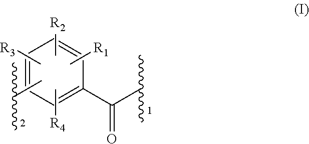
(in the chemical formula (I),
R₁, R₂, R₃ and R₄ are independently —F, —H, —Cl, —Br, —SO₂NH₂, —CF₃, —SO₂CH₃ or —NO₂, respectively,
a wave line 1 represents a covalent bond with the N-terminal amino acid residue of the peptide backbone, and
a wave line 2 represents a covalent bond with the thiol group-containing residue in the peptide backbone).

4. The peptide compound according to claim 2 or 3, wherein the benzoic acid derivative linker is a compound represented by the following chemical formula (II) or (III):
[Chemistry 3] 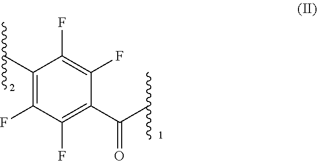 [SMILES: *C(=O)c1c(F)c(F)c(*)c(F)c1F]
[Chemistry 4]

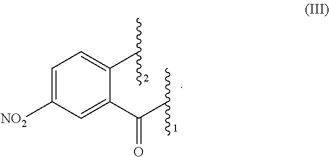 [SMILES: *C(=O)c1cc([N+](=O)[O-])ccc1*]

5. The peptide compound according to any one of claims 1 to 4, wherein the peptide backbone has an amino acid sequence described in any one of SEQ IDs: 4 to 35 and 66 to 77.

6. The peptide compound according to any one of claims 1 to 5, wherein the thiol group-containing residue is a cysteine residue.

7. A pharmaceutical composition for treating or preventing a disease caused by a predetermined compound by binding the peptide compound according to any one of claims 1 to 6 to the predetermined compound.

8. The pharmaceutical composition according to claim 7, wherein the predetermined compound is a bioactive protein.

9. The pharmaceutical composition according to claim 8, wherein the bioactive protein is PCSK9 or IL-5.

10. The pharmaceutical composition according to claim 9, wherein the disease is hypercholesterolemia or allergic disease.

11. An initiator tRNA having a fluorobenzoic acid derivative linker precursor represented by the following chemical formula (IV):
[Chemistry 5] 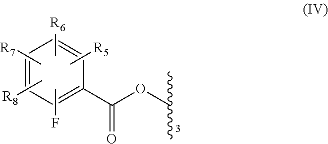
(In the chemical formula (IV),
R₅, R₆, R₇ and R₅ are independently —F, —H, —Cl, —Br, —SO₂NH₂, —CF₃, —SO₂CH₃ or —NO₂, respectively, and
a wave line 3 represents a covalent bond with tRNA).

12. The initiator tRNA according to claim 11, wherein the fluorobenzoic acid derivative linker precursor is a compound represented by the following chemical formula (V) or (VI):
[Chemistry 6] 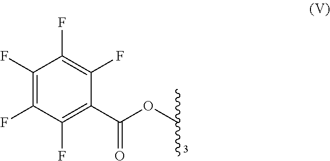 [SMILES: *OC(=O)c1c(F)c(F)c(F)c(F)c1F]
[Chemistry 7]

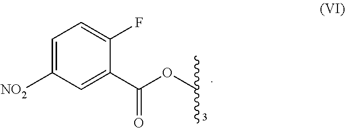 [SMILES: *OC(=O)c1cc([N+](=O)[O-])ccc1F]

13. A method for producing the peptide compound according to claims 1 to 6, comprising:
a providing step for providing at least one type of mRNA; and
a translation step for translating the mRNA in a presence of the initiator tRNA according to claim 11 or 12,
wherein the mRNA has an upstream nucleotide sequence having a nucleotide sequence corresponding to a start codon and a downstream nucleotide sequence having a nucleotide sequence encoding the peptide backbone.

14. A method for screening a target protein, comprising:
a providing step for providing at least one type of mRNA;
a translation step for translating the mRNA in a presence of the initiator tRNA according to claim 11 or 12 and a puromycin DNA linker to obtain a conjugate of a peptide compound having a cyclic moiety with a nucleic acid;
a contact step for contacting the conjugate with the target protein; and
an analysis step for analyzing a binding of the target protein and the conjugate;
wherein the mRNA has an upstream nucleotide sequence having a nucleotide sequence corresponding to a start codon and a downstream nucleotide sequence having a nucleotide sequence encoding the peptide backbone.
